(19) United States
(12) Patent Application Publication
(10) Pub. No.: US 2019/0099085 A1
(43) Pub. Date: Apr. 4, 2019

(54) METHODS AND APPARATUS FOR MONITORING HEART IMPULSES

(71) Applicant: Canform Medical Ltd., Vancouver (CA)

(72) Inventor: John KOBLANSKI, Vancouver (CA)

(21) Appl. No.: 15/725,198

(22) Filed: Oct. 4, 2017

(51) Int. Cl. A61B 5/024; A61B 5/00; A61B 5/11

(52) U.S. Cl. CPC A61B 5/024; A61B 2562/0219; A61B 5/11; A61B 5/742

(57) ABSTRACT

Methods and apparatus for monitoring the heart motion of a subject employ a probe which can be coupled to a portion of the anatomy of the subject, such as the thyroid cartilage of the subject. The probe is mounted on a lever supported by an elastic fulcrum. The probe is biased toward the subject by the elastic fulcrum. The probe detects movements caused by the heart motion. Such movement may be amplified by the lever arm which is pivotable about the longitudinal axis of the elastic fulcrum. A motion sensing apparatus may be provided at or near a distal end of the lever arm to monitor motions of the heart of the subject.

### TECHNICAL FIELD

This invention relates to methods and apparatus for non-invasively monitoring heart forces. The methods and apparatus are useful for the non-invasive monitoring of cardiac functions, in particular, but not exclusively, of human hearts.

### BACKGROUND

Heart disease is a major cause of mortality. There is a need for methods and apparatus that will permit early detection of problems of the heart and circulatory system and for methods and apparatus capable of yielding information useful for diagnosing conditions of the heart and circulatory system.

Measurement of the heart's motions and/or accelerations (commonly referred to as impulse cardiography), provides valuable insights into the condition of the heart. When the heart is beating, the heart mass—including blood contained in the heart—exerts forces on surrounding tissues. This results in a change in momentum of the heart mass. The changes of the momentum of the heart mass with time can be a good indicator of heart function. In particular, heart abnormalities can cause the pattern and the amplitude of the momentum to change. Therefore information characterizing the motions of a person's heart has diagnostic value.

The heart generates both strong and weak forces, which can all have diagnostic significance. Methods and apparatus which enable the measurement of both systolic and diastolic phases of the heart cycle are desirable. For example, it is desirable to obtain measurements that characterize the isovolumetric phase (i.e. the heart's contraction before the blood ejection) of a subject's heart cycle. Isovolumetric contraction is strongly correlated to the ejection phase in magnitude and duration. As an example of a valuable diagnostic result that can be obtained by monitoring heart motion, a large force of contraction in the isovolumetric phase combined with a low-magnitude ejection is a strong indicator that stenosis of the aortic valve exists.

Prior methods for non-invasive monitoring of cardiac function have included:
Mechanical methods, such as pulse recording of the jugular carotid artery or apex cardiography.
Electrical techniques, such as electrocardiograms (ECGs).
Imaging techniques, including echocardiology, radiography and magnetic resonance imaging (MRI).

Existing mechanical methods can be inaccurate because of physical differences between subjects. For example, the intensity of heart sounds is not a good measure of heart function because physiological differences between subjects, such as differences in thickness of layers of fat in the subjects, affect the intensity of heart sounds.

Electrical techniques suffer from the disadvantage that it is difficult to correlate the measured electrical signals with the force of cardiac contraction. Imaging techniques are also subject to this problem. For example, an echocardiogram determines a ratio known as the “ejection fraction”. In a normally-functioning heart, the ejection fraction may be related to the force of the heart's contraction. However, if the heart is not functioning normally, then this relationship may fail to hold true.

None of the above-mentioned prior methods or techniques can accurately characterize the isovolumetric phase of the heart cycle. Characteristics of the isovolumetric phase can be important in identifying coronary artery disease and other heart-related conditions.

Miniature accelerometers within a stethoscope were evaluated as one feasible solution by Pinchak, ESOPHAGAEL ACCELERATION AND THE CARDIOVASCULAR SYSTEM, Journal of Sound and Vibration, 1979, pp. 369-373.

Koblanski, U.S. Pat. No. 5,865,759, discloses an apparatus and method for assessing cardiac function in human beings. The apparatus provides a sensing mechanism positioned on the thyroid cartilage in the neck against the trachea for sensing a response of the thyroid cartilageo heart function. A restraining system is provided to hold the sensing mechanism in position.

Koblanski, U.S. Pat. No. 8,116,858, discloses additional apparatus and methods for assessing cardiac function in human beings. In one embodiment, the apparatus provides a probe attached to a pivotable lever to amplify motion of the thyroid cartilage. Motion of a distal end of the probe is measured by an accelerometer. The probe may be biased against the thyroid cartilage of the subject by a bias spring.

There is a general desire for improved apparatus and methods for monitoring heart motions.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a heart monitoring apparatus having an elastic fulcrum spanning between first and second portions of a support member, an elongated lever arm attached to the elastic fulcrum, the lever arm having first and second ends, a probe at a first end of the lever arm, the probe couplable to a thyroid cartilage of a subject and biased toward the thyroid cartilage by the elastic fulcrum, and

wherein the lever arm is pivotable about the longitudinal axis of the elastic fulcrum in response to movements of the subject's thyroid cartilage.

In some embodiments, a transverse width of the first end of the lever arm is greater than a transverse width of the second end of the lever arm. A portion of the lever arm that contacts the elastic fulcrum may have a transverse width that is greater than the transverse width of the second end of the lever arm. The probe may have a transverse width that is approximately equal to the transverse width of the portion of the lever arm that contacts the elastic fulcrum. The probe may have a cross-sectional shape that is the same as a cross-sectional shape of the portion of the lever arm that contacts the elastic fulcrum. In some embodiments, the probe comprises a flat blade portion and the flat portion is couplable to the thyroid cartilage of the subject by abutting the flat portion against the thyroid cartilage of the subject.

In some embodiments, The elastic fulcrum comprises two or more elastic cables twisted about one another. The two or more elastic cables twisted about one another may be a single elastic cable folded over itself lengthwise. The lever arm may be inserted between two of the two or more elastic cables twisted about one another. The two or more elastic cables may bias the lever arm in a first direction of rotation about the elastic fulcrum

In some embodiments, the lever arm is attached to the elastic fulcrum by a releasable clamping device.

In some embodiments, a releasable brake mechanism is provided. The releasable brake mechanism may include a clamp arranged to clamp the lever arm to prevent movement of the lever arm relative to the support member.

In some embodiments, the support is U-shaped. A first end of the elastic fulcrum may be passed through a first aperture defined by the first portion of the support member thereby attaching the first end of the elastic fulcrum to the first portion of the support and a second end of the elastic fulcrum may be passed through a second aperture defined by the second portion of the support member thereby attaching the second end of the elastic fulcrum to the second portion of the support.

In some embodiments, a motion sensing apparatus is attached to the lever arm between the elastic fulcrum and the second end of the lever arm. The motion sensing apparatus may include an accelerometer and wherein the accelerometer is attached to a power source by a cable that runs from the lever arm to the support member along at least a portion of the elastic fulcrum. The motion sensing apparatus may include a light source and wherein the light source is directed at a screen for monitoring movement of the lever arm. The motion sensing apparatus may include a mirror for reflecting a light source toward a screen for monitoring movement of the lever arm.

In some embodiments, a distance between the first end of the lever arm and the elastic fulcrum is less than a distance between the elastic fulcrum and the second end of the lever arm. A first length of the elastic fulcrum between the first portion of the support member and the elongated lever arm may be greater than a second length of the elastic fulcrum between the second portion of the support member and the elongated lever arm.

In some embodiments, the apparatus includes a display for displaying a first line representing motion of the second end of the lever arm in response to movements of the subject's thyroid cartilage, and a second line representing a concurrent electrocardiogram, wherein the first line and the second line are aligned temporally. In some embodiments, apparatus is configured to identify an isovolumetric phase of the first line as a portion of the first line that occurs 83 milliseconds after a QRS phase of the second line.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A is a schematic front view of a heart monitoring apparatus according to one embodiment of the invention.

FIG. 1B is a schematic front view of a heart monitoring apparatus according to another embodiment of the invention.

FIG. 10 is an elevated view of a heart monitoring apparatus according to another embodiment of the invention.

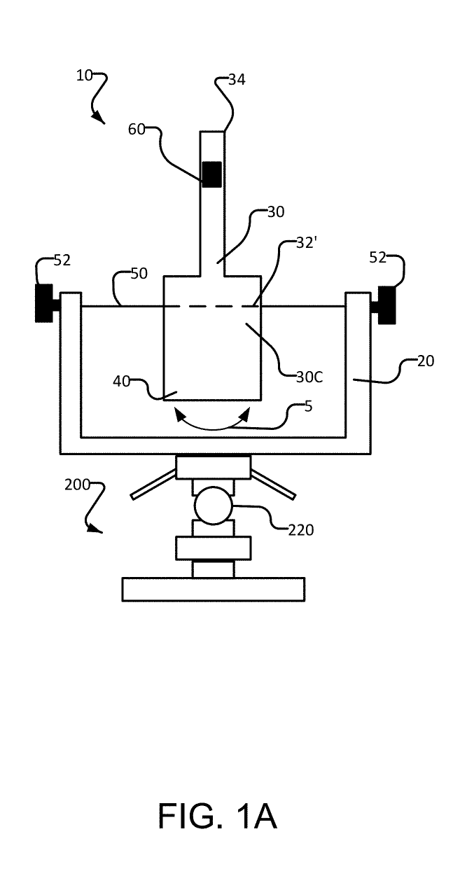

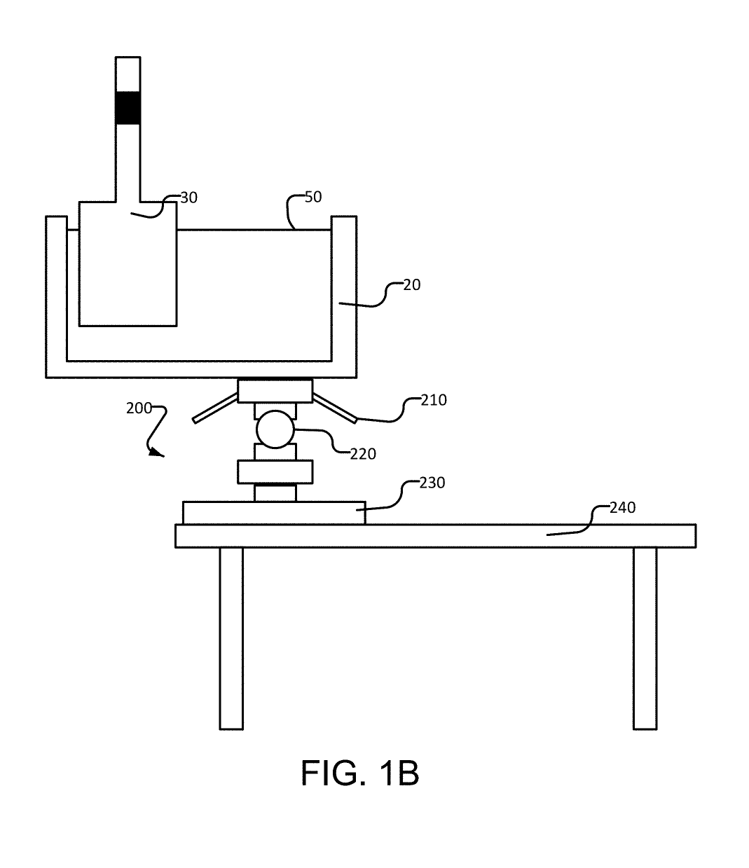

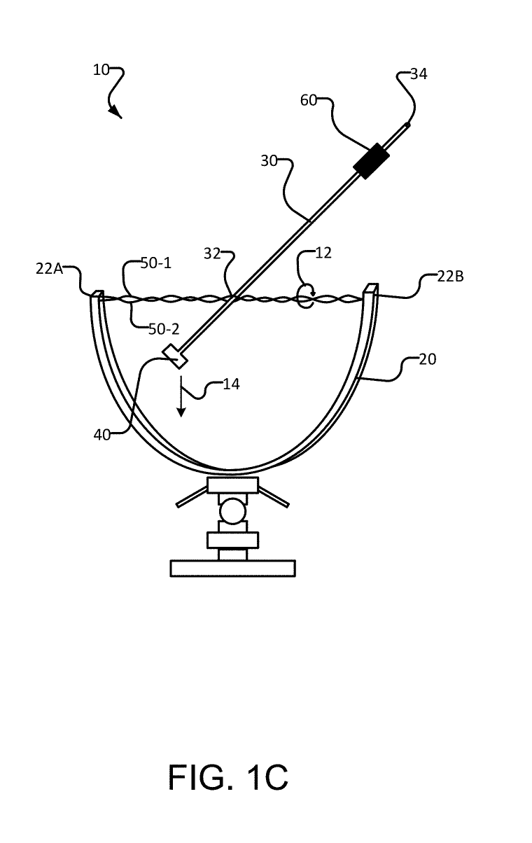

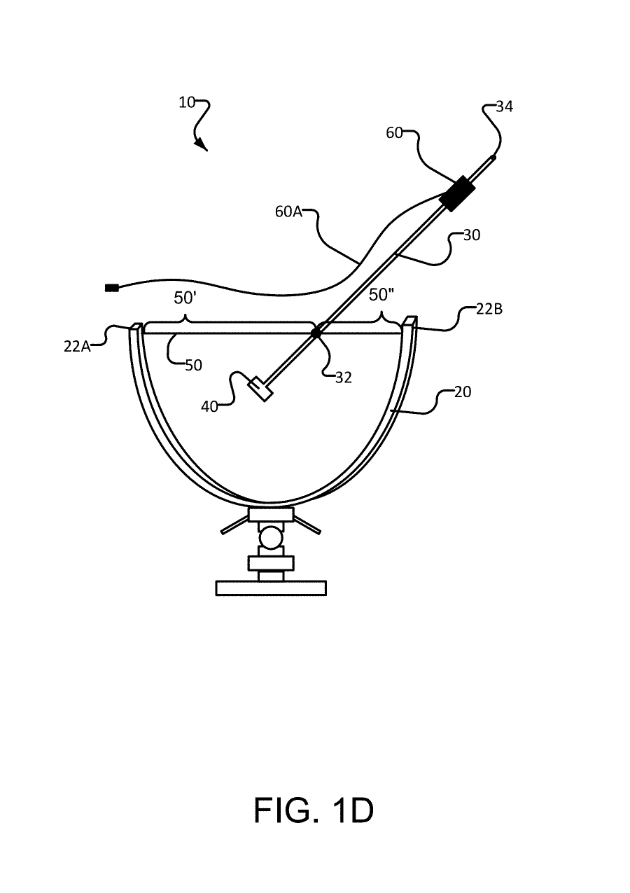

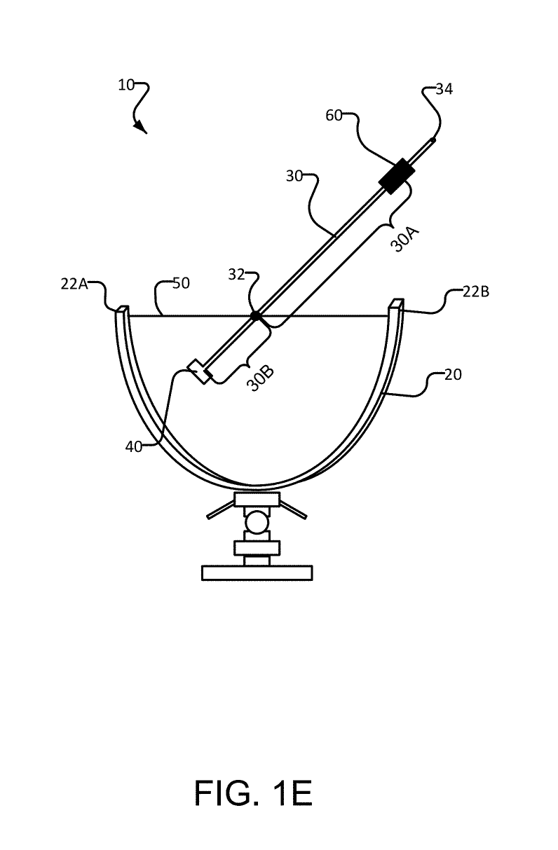

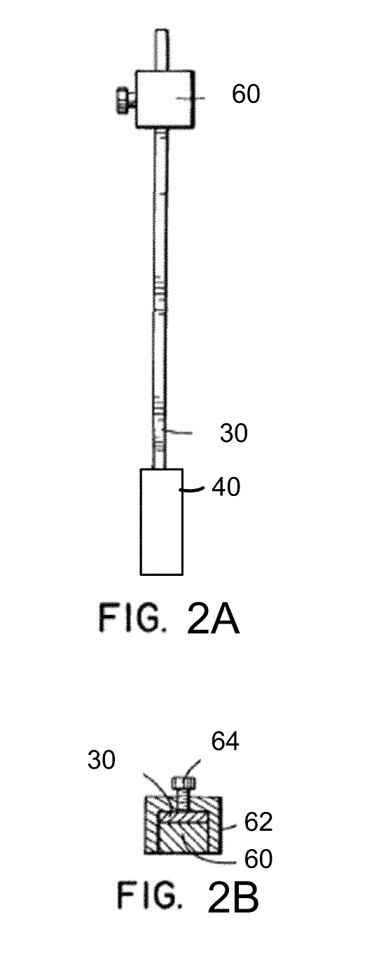

FIG. 1D is an elevated view of a heart monitoring apparatus according to another embodiment of the invention.

FIG. 1E is an elevated view of a heart monitoring apparatus according to another embodiment of the invention.

FIG. 2A is a side view of a lever having a probe and accelerometer.

FIG. 2B is a cross-sectional view of the lever and accelerometer of FIG. 2A.

FIG. 3 is a perspective view of a heart monitoring apparatus according to one embodiment of the invention.

FIG. 4 is a schematic view of a display depicting measurements from a heart monitoring apparatus and an ECG according to one embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

One aspect of the invention provides an apparatus for monitoring motions of a subject's heart. The apparatus comprises a probe that can be placed against an anatomical structure (e.g. the thyroid cartilage) of the subject which moves in response to motions of the heart. The probe may be biased against the anatomical structure by an elastic fulcrum. The elastic fulcrum maintains relatively constant pressure of the probe against the subject despite movement of the subject relative to the apparatus. In some embodiments, the probe is connected to a motion sensor (e.g. an accelerometer) by a mechanical amplifier that amplifies the motion of the probe to improve readings of the motion sensor. Readings of the motion sensor may be analyzed by a medical professional to detect and diagnose problems of the heart and circulatory system.

FIG. 1A depicts a heart monitoring apparatus 10 according to one non-limiting example embodiment of the invention. Heart monitoring apparatus 10 comprises a probe 40 arranged at one end of a lever arm 30. Lever arm 30 is pivotable about an elastic fulcrum 50 at fulcrum point 32. Elastic fulcrum 50 is supported by support 20.

Probe 40 provides a connection between the subject and apparatus 10. Probe 40 may be configured to engage the upper edge of a subject's thyroid cartilage. Probe 40 may comprise a flat blade (similar to the end of a ruler), a rounded end of a rod, or a hollow end of a tube, etc. In some embodiments, probe 40 is provided by an extension of the cross-sectional shape of at least a portion of Lever arm 30. Such a shape may improve the ease with which probe 40 engages cartilage T, without needing to align probe 40 side-to-side. Such a shape may also reduce sensitivity of probe 40 to a pulse of a nearby artery or the like.

Probe 40 may mount to a first end of Lever arm 30. In some embodiments, probe 40 is provided by part of Lever arm 30 or an extension of Lever arm 30. Probe 40 may comprise a soft coating (e.g. rubber, silicone, or the like) to increase the comfort of the subject and improve coupling with the thyroid cartilage of a subject.

In some embodiments, probe 40 is detachable from Lever arm 30. In this way, various differently-shaped (and/or cleaned) probes can be installed on Lever arm 30 to ensure a proper fit between the thyroid cartilage of the subject and probe 40. Some probe 40 attachments may be longer, thereby decreasing the mechanical amplification by Lever arm 30 while others are shorter thereby increasing the mechanical amplification by Lever arm 30. In some embodiments, probe 40 is attached to Lever arm 30 by a snap-fit connection, screws (e.g. plastic or metal screws), press-fit or any suitable fitting.

By improving coupling between probe 40 and the thyroid cartilage, it may be possible to employ heavier (e.g. less expensive and more readily available) sensors (e.g. accelerometer 60) without reducing the functionality of apparatus 10. Similarly, by improving coupling between probe 40 and the thyroid cartilage, it may be possible to employ heavier or thicker elastic fulcrums which may also serve to reduce costs. Furthermore, by improving coupling between probe 40 and the thyroid cartilage, it may be possible to employ different types of sensors, such as for example, variable resistors and/or variable capacitors in place of or in addition to accelerometer 60. Furthermore, by improving coupling between probe 40 and the thyroid cartilage, it may be easier to operate apparatus 10 to obtain accurate and measurable movement of distal end 34 of Lever arm 30 in response to heart motions.

Lever arm 30 may serve to provide adjustable mechanical amplification of motions of probe 40. Adjustable mechanical amplification allows for calibration of apparatus 10 to thereby largely eliminate inter-instrument differences, and enable comparison of data results between similar apparatus 10 at different centres of medical treatment, diagnosis or research. Various methods of calibration are described further herein. The mechanical amplification provided, for example, by Lever arm 30, can contribute to improved signal-to-noise ratios in comparison to apparatus that provides only electrical amplification of the signal output by an accelerometer.

Lever arm 30 may comprise a relatively rigid elongated member having any suitable cross section. For example, Lever arm 30 may be round in cross section (e.g. circular, oval, etc.) or polygonal (e.g. rectangular, square, triangular, etc.). To reduce the mass of Lever arm 30, Lever arm 30 may be hollow, although this is not mandatory.

Lever arm 30 may comprise any suitable material. Lever arm 30 may comprise a lightweight material. For example, Lever arm 30 may comprise wood (e.g. balsa wood), metal, polymer, composite, carbon fiber, fiber reinforced polymer, fiberglass, etc. In some embodiments, Lever arm 30 is sufficiently long that it cannot be rotated about elastic fulcrum 50 without contacting support 20 (e.g. to prevent unwanted twisting or untwisting of elastic fulcrum 50). In some embodiments, padding is provided on support 20 to protect Lever arm 30 or a sensor (e.g. accelerometer 60) located on Lever arm 30 from damage due to contact with support 20.

In the illustrated embodiments, Lever arm 30 is depicted as being relatively straight. This is not necessary. In other embodiments, Lever arm 30 may be curved or bent. Lever arm 30 may be any suitable length. In some embodiments, Lever arm 30 is in the range of 6 to 15 inches (about 15 to 38 cm) long.

As depicted in FIG. 1E, Lever arm 30 may be nominally split into a load section 30A and an effort section 30B. Load section 30A comprises the section of Lever arm 30 between fulcrum point 32 and distal end 34. Effort section 30B comprises the section of lever between fulcrum point 32 and probe 40. If the load and effort sections 30A, 30B of Lever arm 30 are unequal in length, then distal end 34 will move more or less than probe 40 in response to motions of the thyroid cartilage. For example, when load section 30A is longer than effort section 30B, Lever arm 30 amplifies the movements of probe 40 caused by movements of the subject's anatomy.

Lever arm 30 may be attached to elastic fulcrum 30 at fulcrum point 32 in various ways. In some embodiments, a clamp is mounted to elastic fulcrum 50 to allow for the attachment point along the length of Lever arm 30 to be adjusted. For example, the ratio of load section 30A to load section 30B may be adjusted by moving the clamp along the length of Lever arm 30. In this way, the amplification effect of Lever arm 30 may be adjusted as desired. In another embodiment, the clamping mechanism is fixedly attached to Lever arm 30 and may be releasably attached to elastic fulcrum 50. Alternatively, Lever arm 30 may be tied between two portions of elastic fulcrum 50, attached to elastic fulcrum 50 by adhesive, or elastic fulcrum 50 may pass through an aperture in Lever arm 30 and be, for example, held in place along elastic fulcrum 50 by stops on either side of Lever arm 30.

In some embodiments, Lever arm 30 is not of constant cross-section and may comprise a portion 30C having a relatively greater transverse width, as depicted in FIG. 1A. In some embodiments, portion 30C is in contact with elastic fulcrum 50 and has a greater transverse width than a distal end 34 of Lever arm 30 and/or any other portion of Lever arm 30. In this way, instead of merely being attached to elastic fulcrum 50 at fulcrum point 32, Lever arm 30 is attached to elastic fulcrum 50 along the length of fulcrum line 32′ which provides a larger contact area than fulcrum point 32 on its own. Portion 30C may be of any suitable transverse width that is greater than the transverse width of at least another portion of Lever arm 30. In some embodiments, portion 30C may be up to four inches in transverse width. The widened portion 30C of Lever arm 30 may prevent unwanted twisting of Lever arm 30 in the directions indicated by double-headed arrow 5, thereby allowing for more repeatable and more accurate readings obtained from apparatus 10 through better coupling of probe 40 with the thyroid cartilage.

Elastic fulcrum 50 supports Lever arm 30 to pivot. As depicted in FIG. 1A, elastic fulcrum 50 spans between first support portion 22A and second support portion 22B of support 20. The spacing between first support portion 22A and second support portion 22B, which determines the length of elastic fulcrum 50, may be of any suitable dimension. In some embodiments, the spacing may be at least as large as the width of the neck of a subject to allow suitable pressure to be applied by probe 40 on the thyroid cartilage without interference by support 20.

Elastic fulcrum 50 may comprise a string, wire, cable or the like that allows Lever arm 30 to pivot freely about the longitudinal axis of elastic fulcrum 50. In some embodiments, elastic fulcrum 50 itself twists to allow Lever arm 30 to pivot. In some embodiments, Lever arm 30 pivots without twisting elastic fulcrum 50. For example, elastic fulcrum 50 may comprise an elastomeric material. In some embodiments, elastic fulcrum 50 is pre-twisted such that it biases probe 40 in a downward direction. Elastic fulcrum 50 may be attached to support 20 by a screw (e.g. similar to that used on a guitar), by a clamp, by a hook, by a clasp, by crimping it, by tying it around a support portion of support 20, by tying it through an aperture in support 20, by adhesive, or by any other suitable mechanism.

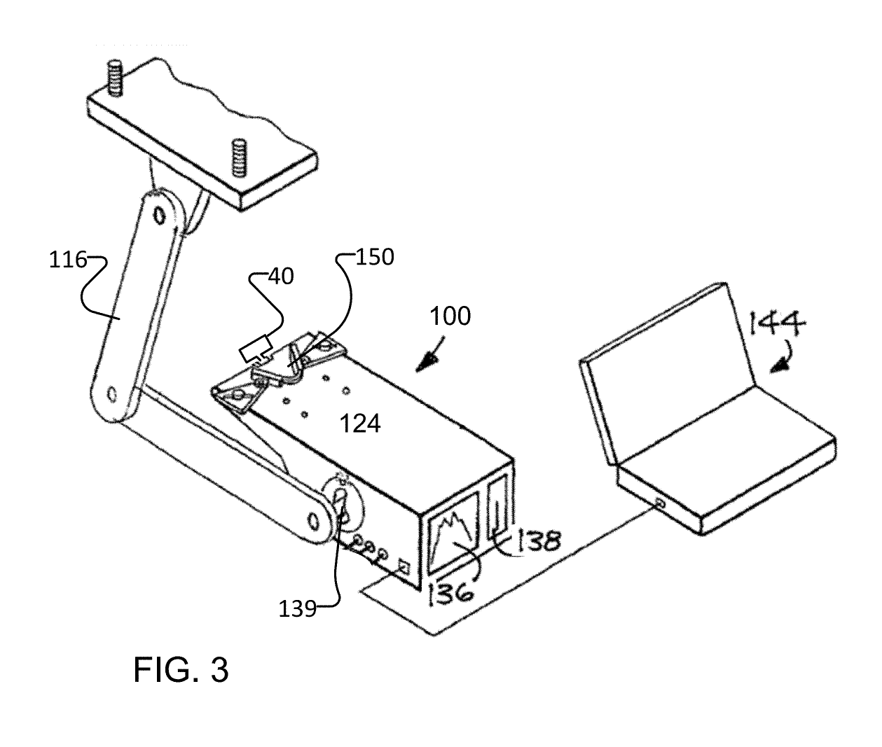

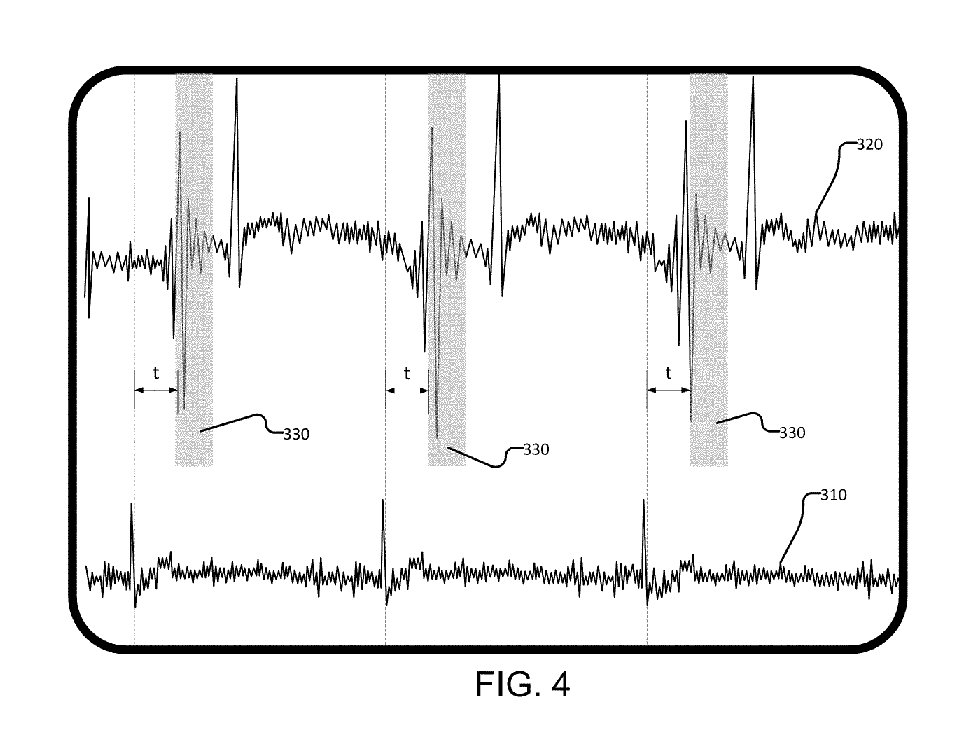

In some embodiments, elastic fulcrum 50 comprises a plurality of elastic cords (e.g. thin bungee cords, or another suitable elastic cord) twisted about one another. For example, FIG. 10 depicts a pair of elastic cords 50-1, 50-2 twisted about one another. Elastic cords 50-1, 50-2 may be twisted any suitable number of times and in any direction. In some embodiments, elastic cords 50-1, 50-2 are twisted in direction 12 such that elastic cords 50-1, 50-2 bias probe 40 downward (i.e. in direction 14 in FIG. 10). The force of the bias on probe 40 may be increased as desired by increasing the number of twists of elastic cords 50-1, 50-2. As can be seen from FIG. 10, Lever arm 30 may optionally be inserted between elastic cords 50-1, 50-2 to frictionally engage Lever arm 30 between elastic cords 50-1, 50-2. In this way, the positioning of Lever arm 30 may be adjusted by sliding it fore and aft between elastic cords 50-1, 50-2. An additional securing element may be employed to hold Lever arm 30 in place between elastic cords 50-1, 50-2. In some embodiments, elastic cords 50-1, 50-2 comprise a single cord folded over itself lengthwise. Elastic cords 50-1, 50-2 may be attached to support 20 in any suitable way as described herein. In some embodiments, elastic cords 50-1, 50-2 pass through one or more apertures in each of support portions 22A, 22B.

Elastic fulcrum 50 may be kept under tension. Tension of elastic fulcrum 50 may cause probe 40 to exert force on the thyroid cartilage of a subject when apparatus 10 is moved toward the subject. The tension of elastic fulcrum 50 may be adjustable by one or more tensioners. For example, FIG. 1A depicts pre-load adjustment knobs 52 which can change tension on elastic fulcrum 50. Pre-load adjustment knobs 52 may serve to adjust a number of twists of elastic fulcrum 50 and/or stretch elastic fulcrum 50 (e.g. in a way similar to that of a guitar tuning key). Tension of elastic fulcrum 50 may be adjusted such that Lever arm 30 is capable of at least 30° of rotation about elastic fulcrum 50. Due to the flexibility and tension of elastic fulcrum 50, the force on the thyroid cartilage may be maintained relatively constant despite minor (horizontal and/or vertical) movements of apparatus 10 relative to the subject. In this way, elastic fulcrum 50 may improve the mechanical coupling between probe 40 and the thyroid cartilage. This may be especially advantageous if the subject has difficulty holding apparatus 10 still due to unsteady or shaking hands or has trouble sitting still.

In some embodiments, elastic fulcrum 50 may be stretched tight enough between first support portion 22A and second support portion 22B such that it provides some resistance to translational movement of Lever arm 30 (i.e. by stretching of elastic fulcrum 50). Elastic fulcrum 50 may cause probe 40 to be biased into and against the notch of the thyroid cartilage. Elastic fulcrum 50 may be stretched between first support portion 22A and second support portion 22B such that it does not resist pivoting of Lever arm 30 (i.e. by twisting of elastic fulcrum 50). Accordingly, apparatus 10 may exhibit increased sensitivity to movement of the thyroid cartilage due to minimal friction resisting pivoting of Lever arm 30.

Support 20 serves to support elastic fulcrum 50. As depicted in FIG. 10, support 20 may comprise a rounded U-shaped structure allowing elastic fulcrum 50 to span between first support portion 22A and second support portion 22B. In other embodiments, support 20 may comprise a squared or somewhat squared U-shaped structure such as depicted in FIG. 1A. In some embodiments, support 20 is V-shaped, H-shaped, Y-shaped, U-shaped or may comprise another shape that is suitable to support elastic fulcrum 50. Support 20 may be made of a relatively rigid material such as metal, polymer or composite, fiber reinforced polymer, wood, etc. although any suitable material may be employed.

Support 20 may be handheld and/or portable and/or mounted to a support structure. A handle may be held by the subject or a medical professional. Alternatively, the handle may be attached to a stationary support such as a tripod or overhead carriage and swivel mechanism. When handheld, the subject may have control over the pressure exerted on their thyroid cartilage by probe 40 thereby allowing them to relieve any discomfort quickly and easily. Such control may also be exercised by the subject moving their body relative to apparatus 10, although this may not always be practical. Despite the ability for the subject to move apparatus 10, the elastic nature of elastic fulcrum 50 maintains sufficient pressure on the thyroid cartilage to maintain coupling between probe 40 and the thyroid cartilage for a range of positions of support 20.

Alternatively, support 20 may be attached to a stationary support 200, as depicted in FIGS. 1A and 1B. For example support 20 may be attached to a, desk, a table, an overhead carriage and swivel mechanism of a type similar to that employed to support overhead lamps of the types used in operating theaters or in dentists' offices, such as is depicted in FIG. 3.

FIGS. 1A and 1B depict apparatus 10 being supported by a base 200. Base 200 comprises one or more pivotal members 220 to allow apparatus 10 to be pivoted toward or away from the subject or to be aligned with the subject. Base 200 may comprise one or more handles 210 to assist in positioning apparatus 10 relative to the subject. Base 200 may be supported by a stand 230 which may be placed on a table, the floor or another suitable surface. In some embodiments, stand 230 may be attached to a table 240 (as in FIG. 1B) or the like by one or more bolts or screws. Base 200 may be employed with a user in a seated position or in a standing position. If the user is in the seated position, the chair may have adjustable height to aid in lining up the user with apparatus 10.

Motion of distal end 34 of Lever arm 30 may be monitored in various ways. In some embodiments, an accelerometer 60 is mounted on Lever arm 30. Accelerometer 60 may be mounted by glue, one or more screws, one or more releasable mechanisms, by friction, by clamping, etc. For example, accelerometer 60 may be located at or near distal end 34 of Lever arm 30. A position of accelerometer 60 along Lever arm 30 may be adjustable.

Accelerometer 60 may comprise any suitable accelerometer such as, for example, an integrated circuit piezoelectric sensor. As depicted in FIGS. 3A and 3B, accelerometer 60 may be fixed to a U-shaped bracket 62. A locking mechanism 64 is provided for releasably securing bracket 62 and, therewith, the accelerometer 60 at a desired position along Lever arm 30. The position of accelerometer 60 along Lever arm 30 can be adjusted by loosening locking mechanism 64 and sliding bracket 62 to a desired position along Lever arm 30. The amount of amplification of the movement of probe 40 can be adjusted by sliding accelerometer 60 to a desired position along Lever arm 30. This adjustment may be used to compensate for the fact that different accelerometers tend to produce different electrical outputs for the same acceleration. Accelerometer 60 may be mounted at such a position along Lever arm 30 that it produces a desired output when Lever arm 30 is moved with a specified acceleration. In other embodiments, accelerometer is fixedly mounted to Lever arm 30 and is not adjustable. Accelerometer 60 may be wired or wireless.

In some embodiments, wires 60A connected to accelerometer 60 run along elastic fulcrum 50. For example, in embodiments where elastic fulcrum 50 comprises twisted cables (such as depicted in FIG. 10) wires 60A could be twisted with elastic cables 50-1, 50-2. Alternatively, wires 60A may be separate from elastic fulcrum 50 as shown in FIG. 1D. To minimize any interference by wires 60A on the sensitivity of apparatus 10, Lever arm 30 may be offset along elastic fulcrum 50, such that a first length of elastic fulcrum 50′ between support portion 22A and fulcrum point 32 is greater than a second length 50″ between support portion 22B and fulcrum point 32. A wire carrying signals from accelerometer 60 may extend generally parallel to first length 50′. In other embodiments, Lever arm 30 may be offset along elastic fulcrum 50 to accommodate mounting apparatus 10 on a particular surface such as a table, as depicted in FIG. 1B. In some embodiments, wires 60A may be looped or have extra slack to minimize the effect of wires 60A on movement of Lever arm 30 (see, for example, FIG. 1D). In some embodiments, a light co-axial cable is integrated into elastic fulcrum 50.

In some embodiments, elastic fulcrum 50 may include conductors connected to transmit power or data to and/or from accelerometer 60.

Apparatus 10 may be calibrated by coupling Lever arm 30 to a vibrator or shaker table that provides a predetermined acceleration and/or amplitude. The position of accelerometer 60 along Lever arm 30 can be adjusted until the output of the output signal of accelerometer 60 has a desired value. In addition or in the alternative, the ratio of load section 30A to effort section 30B can be adjusted until the output of the output signal of accelerometer 60 has a desired value. In some embodiments, a counterweight may be attached to Lever arm 30. The position of the counterweight along the length of Lever arm 30 may be adjustable to allow for calibration of apparatus 10.

In some embodiments, one or more of a variable resistor and a variable capacitor (or any other transducer), that are connected to provide an output signal that varies with motion or angular displacement of Lever arm 30, may be employed instead of or in addition to accelerometer 60. By monitoring change in resistance and/or capacitance, a pattern of displacement may be determined. The displacement pattern produced may yield information sufficient to identify if a phase of the heartbeat (and which phase of the heartbeat) is missing as well as the direction of the pattern thereby enabling the detection of, for example, paradoxical left ventricular motion.

In some embodiments, Lever arm 30 may also or alternatively comprise a mirror for reflecting a light beam from a light source, such as a laser, onto a screen, which forms part of a display. The screen may be a phosphorescent screen of long duration or a position sensing diode array, which provides a digital output that indicates the deflection of the beam by the mirror. The magnitude of the displacement of the heart function can be observed by watching movement of the light on the screen. In some embodiments, the mirror may be replaced with a light source mounted directly on Lever arm 30. The light source mounted directly on Lever arm 30 may direct a light beam at a screen such that displacement of the heart function can be observed by watching movement of the light on the screen.

When it is desired to measure motion of the subject's thyroid cartilage, support 20 may be manipulated to move probe 40 into contact with the top edge of the subject's the thyroid cartilage. In the FIG. 1A embodiment, the subject or a second person (e.g. a nurse, medical technician or doctor) may position probe 40 by moving base 200. Correct coupling of probe 40 with the subject's the thyroid cartilage will generally be indicated by visible rhythmic pivoting of Lever arm 30 that coincides with the subject's heartbeat. Due to the elastic nature of elastic fulcrum 50, movement (vertical, fore and aft, or side to side) of the subject may be accommodated by apparatus 10 without disturbing the coupling between the thyroid cartilage and probe 40.

FIG. 3 shows details of a heart motion measurement apparatus 100 according to a specific example embodiment of the invention. Measurement apparatus 100 may comprise a housing 124 containing an elastic fulcrum 50 supporting a Lever arm 30 which can pivot about a longitudinal axis of elastic fulcrum 50, similar to the FIGS. 1A and 1B embodiments. A handle 138 is provided on housing 124. Apparatus 100 can be guided into a desired position by manipulating handle 138. Housing 124 is connected to support device 116 by a ball joint 139 so that housing 124 can be tilted as desired. Ball joint 139 may have a lock screw for fixing housing 124 in a desired position relative to support device 116.

A display may be provided on housing 124 or at another convenient location. The display may show information about the status of apparatus 100 as well as information about the motion of the subject's heart as measured by apparatus 100. For example, the display may display a waveform showing the displacement of a subject's heart as a function of time, and may include a displacement magnitude display 136 for displaying the magnitude, or amplitude, of the displacement whose waveform is displayed.

Apparatus 100 may be connected to a computer 144. Computer 144 may comprise a laptop computer, a personal computer, or a computer network. Computer 144 may receive data from apparatus 100. The data may comprise data representing heart motion and may also include other data. The data may be stored, manipulated, displayed or otherwise processed by computer 144. Software may be provided to better study or interpret data generated by apparatus 10 or apparatus 100. For example, the software may be used to correlate the data generated to data from an electrocardiogram or the like. In some embodiments, apparatus 10 or apparatus 100 is connected to a signal detector and an oscilloscope for monitoring the heart's motions.

In some embodiments, a chin rest 150 is supplied to support the chin of the subject while apparatus 100 monitors motion of the subject's the thyroid cartilage. Chin rest 150 may be attached to housing 124 or support 20 or another stationary object. Chin rest 150 may be adjustable to allow for proper contact with the chin of the subject. Chin rest 150 may be removable.

In some embodiments, apparatus 10 or 100 may be employed in conjunction with an ECG. The ECG may be taken concurrently with the measurements of apparatus 10. The ECG may be used to identify an isovolumetric phase of the heart and correlate the isovolumetric phase of the heart to particular movements of Lever arm 30. FIG. 4 depicts an exemplary display 300 which could be employed with any of the apparatuses described herein. Display 300 shows a first line representative of an ECG waveform 310 aligned with a second line representative of a waveform 320 showing the displacement of distal end 34 of apparatus 10 (or another similar apparatus such as those described herein). In some cases, it may be possible to identify the isovolumetric phase 330 of the heart as being the phase that occurs at a time t after the QRS complex, as depicted in FIG. 4. In some embodiments time t is approximately 83 milliseconds. In some embodiments, computer 144 or another computer or software may automatically align the waveforms 310, 320 and/or highlight isovolumetric phase 330 as the phase that occurs at time t after the QRS complex to allow isovolumetric phase 330 to be studied. While FIG. 4 depicts both of waveforms 310 and 320 being shown on display 300, it is not necessary that waveform 310 is depicted. Instead, waveform 310 may be used to automatically identify isovolumetric phase 330 without being shown.

Various heart conditions may then be determined by relating the results of apparatus 10 with an ECG. For example, if the ejection velocity is small, despite a large amplitude at the isovolumetric phase, there may be a restriction of the heart valve. As another example, paradoxical septal motion may be identified by using an ECG to identify which motions of Lever arm 30 represent the isovolumetric phase of the heart. As another example, it may be possible to compute a ratio of Lever arm 30 displacements during the isovolumetric phase to Lever arm 30 displacements during the ejection phase to determine whether or not the heart motions are normal.

In some embodiments, to aid with achieving a proper coupling between probe 40 and the thyroid cartilage of the subject, a brake mechanism may be employed to prevent Lever arm 30 from pivoting or moving generally and/or to damp motions of probe 40 while setting up apparatus 10 or 100. Various techniques may be employed to hold or immobilize Lever arm 30. For example, a clamping mechanism may be attached to support 20 to hold Lever arm 30 and prevent Lever arm 30 from moving. The clamping mechanism may hold Lever arm 30 at fulcrum point 32 or closer toward distal end 34 of Lever arm 30. The clamping mechanism is releasable to allow for movement of Lever arm 30 once coupling between probe 40 and the thyroid cartilage is achieved.

While a number of exemplary aspects and embodiments have been discussed above, those of skill in the art will recognize certain modifications, permutations, additions and sub-combinations thereof. For example, embodiments described herein may implement any of the features described in U.S. Pat. No. 8,116,858, which is hereby incorporated herein by reference. It is therefore intended that the following appended claims and claims hereafter introduced are interpreted to include all such modifications, permutations, additions and sub-combinations as are consistent with the broadest interpretation of the specification as a whole.

1. A heart monitoring apparatus comprising:
an elastic fulcrum spanning between first and second portions of a support member;
an elongated lever arm attached to the elastic fulcrum, the lever arm having first and second ends;
a probe at a first end of the lever arm, the probe couplable to a thyroid cartilage of a subject and biased toward the thyroid cartilage by the elastic fulcrum; and
wherein the lever arm is pivotable about the longitudinal axis of the elastic fulcrum in response to movements of the subject's thyroid cartilage.

2. A heart monitoring apparatus according to claim 1 wherein a transverse width of the first end of the lever arm is greater than a transverse width of the second end of the lever arm.

3. A heart monitoring apparatus according to claim 2 wherein a portion of the lever arm that contacts the elastic fulcrum has a transverse width that is greater than the transverse width of the second end of the lever arm.

4. A heart monitoring apparatus according to claim 2 wherein the probe has a transverse width that is approximately equal to the transverse width of the portion of the lever arm that contacts the elastic fulcrum.

5. A heart monitoring apparatus according to claim 2 wherein the probe has a cross-sectional shape that is the same as a cross-sectional shape of the portion of the lever arm that contacts the elastic fulcrum.

6. A heart monitoring apparatus according to claim 2 wherein the probe comprises a flat blade portion, the flat portion couplable to the thyroid cartilage of the subject by abutting the flat portion against the thyroid cartilage of the subject.

7. A heart monitoring apparatus according to claim 1 wherein the elastic fulcrum comprises two or more elastic cables twisted about one another.

8. A heart monitoring apparatus according to claim 7 wherein the two or more elastic cables twisted about one another comprise a single elastic cable folded over itself lengthwise.

9. A heart monitoring apparatus according to claim 7 wherein the lever arm is inserted between two of the two or more elastic cables twisted about one another.

10. A heart monitoring apparatus according to claim 9 wherein the two or more elastic cables bias the lever arm in a first direction of rotation about the elastic fulcrum.

11. A heart monitoring apparatus according to claim 1 wherein the support member is U-shaped.

12. A heart monitoring apparatus according to claim 1 wherein the lever arm is attached to the elastic fulcrum by a releasable clamping device.

13. A heart monitoring apparatus according to claim 1 comprising a releasable brake mechanism, the releasable brake mechanism comprising a clamp arranged to clamp the lever arm to prevent movement of the lever arm relative to the support member.

14. A heart monitoring apparatus according to claim 1 wherein a first end of the elastic fulcrum is passed through a first aperture defined by the first portion of the support member thereby attaching the first end of the elastic fulcrum to the first portion of the support and a second end of the elastic fulcrum is passed through a second aperture defined by the second portion of the support member thereby attaching the second end of the elastic fulcrum to the second portion of the support.

15. A heart monitoring apparatus according to claim 1 comprising a motion sensing apparatus attached to the lever arm between the elastic fulcrum and the second end of the lever arm.

16. A heart monitoring apparatus according to claim 15 wherein the motion sensing apparatus comprises an accelerometer and wherein the accelerometer is attached to a power source by a cable that runs from the lever arm to the support member along at least a portion of the elastic fulcrum.

17. A heart monitoring apparatus according to claim 15 wherein the motion sensing apparatus comprises a light source and wherein the light source is directed at a screen for monitoring movement of the lever arm.

18. A heart monitoring apparatus according to claim 15 wherein the motion sensing apparatus comprises a mirror for reflecting a light source toward a screen for monitoring movement of the lever arm.

19. A heart monitoring apparatus according to claim 1 wherein a distance between the first end of the lever arm and the elastic fulcrum is less than a distance between the elastic fulcrum and the second end of the lever arm.

20. A heart monitoring apparatus according to claim 1 wherein a first length of the elastic fulcrum between the first portion of the support member and the elongated lever arm is greater than a second length of the elastic fulcrum between the second portion of the support member and the elongated lever arm.

21. A heart monitoring apparatus according to claim 1 comprising a display for displaying:
a first line representing motion of the second end of the lever arm in response to movements of the subject's thyroid cartilage; and
a second line representing a concurrent electrocardiogram;
wherein the first line and the second line are aligned temporally.

22. A heart monitoring apparatus according to claim 21 wherein the apparatus is configured to identify an isovolumetric phase of the first line as a portion of the first line that occurs 83 milliseconds after a QRS phase of the second line.
